(19) United States
(12) Patent Application Publication
(10) Pub. No.: US 2022/0296424 A1
(43) Pub. Date: Sep. 22, 2022

(54) System and Method for Personalized Implantable Scaffolds for Wound Healing

(71) Applicant: Ashwin Prabhakar, Madison, AL (US)

(72) Inventor: Ashwin Prabhakar, Madison, AL (US)

(21) Appl. No.: 17/696,306

(22) Filed: Mar. 16, 2022

Related U.S. Application Data
(60) Provisional application No. 63/162,266, filed on Mar. 17, 2021.

(51) Int. Cl. A61F 13/00; A61B 5/00; G06F 30/23

(52) U.S. Cl. CPC A61F 13/00987; A61B 5/445; G06F 30/23; A61F 2013/00357

(57) ABSTRACT

A system and method for development of personalized implantable scaffolds for wound healing. The process involves 3D scanning or 3D imaging of wounds to create 3D images which are post processed using CAD modeling software to generate 3D models of the wounds for integration with 3D, 4D, 5D, or 6D printers. Using biocompatible materials, scaffolds specific to the wounds create a personalized fit. The scaffolds can be integrated with wound healing components, cells and sensors for real-time monitoring of the local environment to promote healing. The developed personalized scaffolds for wounds have great potential to significantly reduce healthcare costs and patient treatment time.

### CROSS-REFERENCE TO RELATED APPLICATIONS

This patent application claims priority to U.S. Provisional Application No. 63/162,266 filed Mar. 17, 2021, which provisional is incorporated herein by specific reference in its entirety.

### BACKGROUND

Wound treatment and management has become a major healthcare problem affecting more than 7 million people in the United States and costing more than $50 billion annually. Wound healing process comprises of four overlapping phases: Hemostasis, Inflammatory, Proliferative, and Remodeling (Maturation). Wounds can be classified as acute or chronic. An acute wound is expected to progress through the phases of normal healing, resulting in the closure of the wound within 4 weeks. Chronic wounds on the other hand get stuck in one of the phases (most often inflammatory) and may takes months and years to heal and require extensive treatment.

Hemostasis is the first phase during healing and starts at the onset of injury. Hemostasis results in the process of the wound being closed by clotting or coagulation. This is mediated by the interaction of the platelets with the damaged blood vessel. Thrombin initiates the formation of fibrin mesh that strengthens the platelets into a stable clot. This process also results in the initial release of cytokines and growth factors in the wound. The inflammatory phase role is to enter the wound area to destroy bacteria, remove debris and prepare the wound for growth of new tissue. This is mediated by white blood cells comprising of neutrophils, macrophages, and other cells. This phase starts within 24 hours and remains active for 4-7 days. This stage is also the second source of growth factors and cytokines. Elevated inflammation stage is known to delay healing.

During the proliferative phase, granulation tissue composed of fibroblasts, macrophages, blood vessels, and extracellular matrices take over the wound. Basal epithelial cells proliferate and migrate over the granulation tissue to close the wound surface resulting in a scar. Maintenance of a moist wound environment and protection of new tissues is important as any changes from optimal physiological conditions will result in this phase also to be prolonged. The Proliferative phase often lasts up to 21 days. During the maturation phase, the initial scar is removed and replaced by the matrix that is similar to normal skin. The maturation phase varies greatly from wound to wound, often lasting anywhere from 21 days to two years.

There are two types of acute wounds: traumatic/accidental wounds and surgical wounds. Accidental wounds include minor cuts, lacerations, bites, abrasions, burn, etc. A surgical wound is either incised and sutured or kept open to heal by itself following surgery. Common types of chronic wounds include infectious wounds, burn wounds, surgical wounds, and ulcer wounds comprising of venous, arterial, diabetic, and pressure ulcers.

Traditional wound dressing such as gauze and bandages are used for protecting the wound from contaminations with or without the incorporation of antimicrobial agents while promoting healing. However, while they absorb fluid extrusions, they adhere to the wound leading to damage on removal. To overcome these situations, dressings that promote healing in a moist environment (foams, hydrogels, hydrocolloids, etc.) have been developed. However, these dressings are not suitable for treatment of large areas. For these wounds, skin grafts have been used as a possible solution. However, these grafting processes while being expensive are time consuming and also severely limited due to donor sites availability. Allografts have also shown promise due to their readily availability. However, immunological rejection and disease transmission are major concerns. These limitations and concerns have led to the growth of cell based wound treatment.

Cells of the different layers of the skin (keratinocytes from epidermis and fibroblasts from dermis) are isolated or differentiated from stem cells and injected directly to the wound. Additional layers of vascular cells can also be incorporated. However, development of multiple layers of the skin is a challenge with this method. Bioprinting has evolved as an advancement in the field of tissue engineering for developing layers of skin cells similar to in vivo architecture and wound treatments have been demonstrated in animal models. However, all of these studies used expensive instruments in addition to the need for trained personnel, and most importantly are not amenable for use in clinics and hospital with minimal infrastructure. Finally, there is currently no system in the market that can scan large and deep cavity wounds and provide a personalized implantable scaffold for treatment.

### SUMMARY

In some embodiments, a system for preparing a personalized implantable wound healing scaffold can include: a 3D data acquisition unit including at least one of a 3D scanner or 3D imaging device; a 3D model generator operably coupled with the 3D data acquisition unit; and a physical printer operably coupled with the 3D model generator. In some aspects, the 3D model generator includes one or more processors and one or more non-transitory computer readable media storing instructions that in response to being executed by the one or more processors, cause the 3D model generator to perform operations. The operations can include: obtaining 3D data of a wound from the 3D data acquisition unit; and generating a 3D model from the 3D data. In some aspects, the physical printer is selected from a 3D, 4D, 5D, or 6D printer. In some aspects, a 3D mold caster is operably coupled with the 3D model generator. In some aspects, the system includes a sensor for real-time monitoring of the wound, optionally a personalized implantable wound healing scaffold having the sensor, and there is a monitor operably coupled with the sensor for acquiring sensor data, saving the sensor data, and analyzing the sensor data. In some aspects, the 3D model generator is configured to generate 3D models of the wound, and the 3D model is a model of the wound to receive the wound healing scaffold. In some aspects, the 3D model generator is configured to generate 3D models of the wound and/or personalized implantable wound healing scaffold to fit into the wound, and wherein the 3D model is a model of the wound and/or wound healing scaffold adapted to fit into the wound.

In some embodiments, a method of preparing an implantable wound healing scaffold can include: acquiring 3D data of a wound with a 3D acquisition unit that includes at least one of a 3D scanner or 3D imaging device; generating a 3D model of the wound; and forming the implantable wound healing scaffold in accordance with the 3D model of the wound. In some aspects, the step of forming the implantable wound healing scaffold in accordance with the 3D model of the wound includes physically printing, with a 3D, 4D, 5D, or 6D printer, a mold of the wound and/or an implantable wound healing scaffold in accordance with the wound. In some aspects, the step of forming the implantable wound healing scaffold in accordance with the 3D model of the wound includes casting an implantable wound healing scaffold in the mold of the wound.

In some embodiments, the methods can include: generating at least one wound data structure of a point cloud or mesh of the wound; importing the at least one wound data structure of the wound to a CAD software; extracting desired parts of wounds from the imported mesh; and generating a CAD model of the wound.

In some embodiments, the methods can include at least one of: creating of wound mold, which includes a mold of the wound; using the wound mold to develop exact replica of the wound in the form of a wound healing scaffold; integrating at least one agent into the wound healing scaffold; or integrating the wound healing scaffold with sensors for wound environment monitoring.

In some embodiments, a method of treating the sound can include implanting the implantable wound healing scaffold in accordance into the wound.

In some embodiments, the methods can include: monitoring the wound healing scaffold for changes; removal of wound healing scaffold based on observed changes; acquiring new 3D data of the wound by rescanning or reimaging of the wound to develop a second wound healing scaffold corresponding to the new morphology of the healing wound; forming the second wound healing scaffold; and implanting the second sound healing scaffold into the wound.

In some embodiments, the methods can include: resolving the 3D data to mimic the layers of the wound, skin, and the vasculature; integrating cells of different layers of the wound healing scaffold; and forming a wound healing implant with an epidermis layer with keratinocytes, a dermis layer with fibroblasts, a base layer with adipocytes and/or stem cells and a vasculature structure with endothelial cells in the wound healing scaffold, wherein the cells can be natural or synthetically modified cells.

In some embodiments, the methods can include: integrating a therapeutic composition with the wound healing scaffold to treat/prevent infection; and releasing an active agent from the therapeutic composition before, during or after implantation of the scaffold in the wound.

In some embodiments, the methods can include forming the wound healing scaffold with a material with components selected from natural polymers selected from: collagen, gelatin, chitosan, alginate, silk, elastin, laminin, fibronectin, hyaluronic acid, or combinations thereof; or synthetic polymers selected from polyethylene glycol (PEG), polyethylene glycol diacrylate (PEGDA), polycaprolactone (PCL), polyvinyl alcohol (PVA), poly(d,l-lactide-co-glycolide) (PLGA); or native, synthetic, modified collagen methacrylate (ColMA) or gelatin methacrylate, or a combination of those in varying ratio.

In some embodiments, the methods can include: detecting changes in a local wound environment adjacent the wound healing scaffold; transmitting data regarding the changes in the local wound environment actively or passively to a device and/or server and/or app and/or cloud capable of receiving the transmission; and using the data to decide the next course of wound treatment.

In some embodiments, the methods can include performing at least one of: Laser triangulation, Structured light 3D scanning technology, Photogrammetry, Contact-based 3D scanning technology, Laser pulse-based 3D scanning technology, Computerized Tomography, Single-photon emission computed tomography, Magnetic resonance imaging, Ultrasound imaging, or terahertz spectroscopy.

In some embodiments, the methods can include: developing a wound mold for casting or printing scaffolds, and using 3D, 4D, 5D, or 6D printing methods based on inkjet, extrusion, and laser-based technologies, selected from Stereolithography (SLA), Fused Deposition Modeling (FDM), Selective Laser Sintering (SLS), Digital Light Process (DLP), Multi Jet Fusion (MJF), PolyJet, Direct Metal Laser Sintering (DMLS), Electron Beam Melting (EBM), Solvent-based extrusion free forming (SEF), solvent-based extrusion (SBE), Laser-induced forward transfer (LIFT) bioprinting, or a combination of the above.

In some embodiments, the methods include preparing the wound healing scaffold to include at least one of: natural or synthetic DNA, RNA, exosomes, proteins, antibacterial, antifungal, antiviral, bacteriophages, growth factors, cytokines, chemokines and signaling molecules for cell and tissue growth, selected from the group consisting of: EGF, FGF, KGF, TGF-β, PDGF, VEGF, GM-CSF, CTGF, IL-1, IL-6, TNF, CXCL1, CXCL12, CCL2, or combinations thereof; oxygen generation components selected from the group consisting of calcium peroxide, magnesium peroxide, sodium percarbonate, hydrogen peroxide, perfluorodecalin, perflubron, and combinations thereof; oxygen generation components from natural sources; inorganic metal or non-metal particles; or organic polymeric or non-polymeric particles; microparticles; nanoparticles; biodegradable particles. In some aspects, the wound healing scaffold includes magnetic or electrically conductive particles mixed or as a layer on top to provide pulse based therapy.

In some embodiments, the methods can include: determining one or more layers for the wound healing scaffold; preparing the one or more layers with matrix and/or cells; and assembling the one or more layers to form the wound healing scaffold.

In some embodiments, a method for customizing a wound healing scaffold can include: locating a wound; measuring one or more dimensions of the wound or providing a scale reference marker at the wound for imaging; acquiring 3D data of the wound; creating a 3D model of the wound from the 3D data; creating a 3D mold from the 3D model; and creating the wound healing scaffold in the 3D mold.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates the overall process for developing customized scaffolds to fit the wounds.

FIG. 2 illustrates the process for sequential scaffold formation to match the healing wounds.

FIG. 3 illustrates the process for generating multiple layers of scaffold.

FIG. 4 illustrates the process of 3D imaging, 3D printing, and hydrogel based scaffolds.

FIG. 5 shows an example of photogrammetry based 3D imaging and extrusion based 3D printing of wound mold.

FIGS. 6A-6F show examples of photogrammetry based automated 3D imaging, 3D printing of wound molds and implantable scaffold generation with perfect fit to the wounds.

FIG. 7 shows example of 3D model creation from the wounds

FIG. 8 shows examples of wound molds and scaffolds for personalized fit to the wounds.

FIGS. 9A-9B show data for monitoring of drug delivery and temperature changes.

FIG. 10 shows an example computing device.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention includes a system and methodology to develop personalized implantable scaffolds for wound healing. Any 3D imaging or scanning based system can be used to acquire 3D images of deep cavity wounds. The 3D images are post processed to generate 3D models of the wounds. Biocompatible scaffolds are then generated using any of 3D, 4D, 5D, or 6D printing techniques or casting techniques. The scaffolds can be further integrated with therapeutics, organic or inorganic materials and sensors (electrical, optical, magnetic, thermal) to guide wound healing and to monitor the local wound environment and to guide the healing of the wound. Scaffolds can also be integrated with growth factors and cells to match the layers of the wound required for healing. The developed personalized scaffolds for wounds have great potential to significantly reduce healthcare costs and patient treatment time

The system and method can be used for tracking wound healing and to allow sequential treatment similar to changing bandages. Customized scaffold made during each stage of the wound closure will accelerate the process of healing while minimizing infections. The scaffolds can be used for any kind of wounds both acute and chronic and for any part of the body. The scaffolds can also be mixed with different formulations and matrices depending on the wound type, location, and the requirements of the clinician for treatment.

The present invention can generate personalized scaffolds with or without development of wound molds. The wound molds can also be made of biodegradable components for direct implantation into the wound with the scaffolds. The process for development of customized perfect fit scaffolds relies on generation of the exact features of the wound including scale, morphology and desired cellular components. Wound scaffolds can also be tested for release of therapeutics, development of new biomaterials, and real-time monitoring of the local wound environment to guide the healing process.

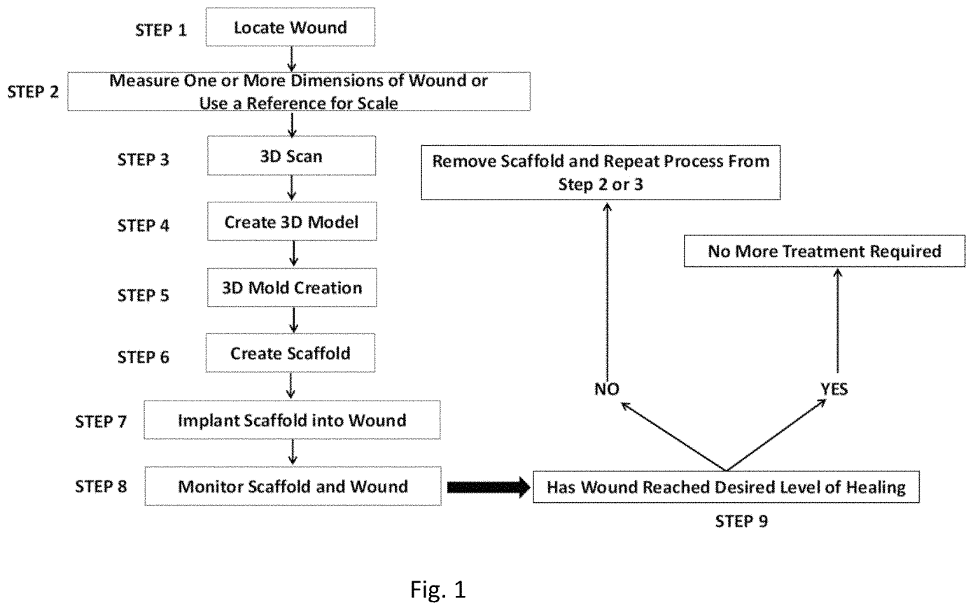

FIG. 1 illustrates a process of the wound scaffold formation using a wound mold. First, the wound is located, next reference scales are taken followed by a 3D scan. A 3D model is developed and the wound molds are generated. Next, scaffolds are created using printing or casting and implanted into the wounds. They are monitored and the process is repeated until no more treatment is required based on the observation of the wound. This process can also be performed without the use of 3D mold where following 3D model development, 3D, 4D, 5D, or 6D printing techniques that allow direct printing can be used.

FIG. 1 shows the summary of the process for developing scaffolds to fit wounds. Step 1) locate wound; Step 2) measure one or more dimensions of wound or use a reference for scale; Step 3) perform a 3D scan of the wound; Step 4) create a 3D model of the wound; Step 5) create a 3D model of the wound; Step 6) create a physical scaffold that fits into the wound and matches the wound; Step 7) implant the scaffold into the wound; Step 8) monitor the scaffold and wound; and Step 9) determine whether or not wound has reached desired level of healing. If the desired level of healing has been reached, then no more treatment is needed or required. If the desired level of healing has not been reached, then the protocol can include removing the scaffold and repeating the process beginning in Step 2 or Step 3. The process can be iterated as needed to treat the wound. That is, multiple sequentially smaller scaffolds may be obtained from the scan and modeling as the wound heals. However, a single scaffold can be sufficient in many instances. For example, a daily or weekly assessment of the wound healing and determination of whether or not a new scaffold is needed can be performed.

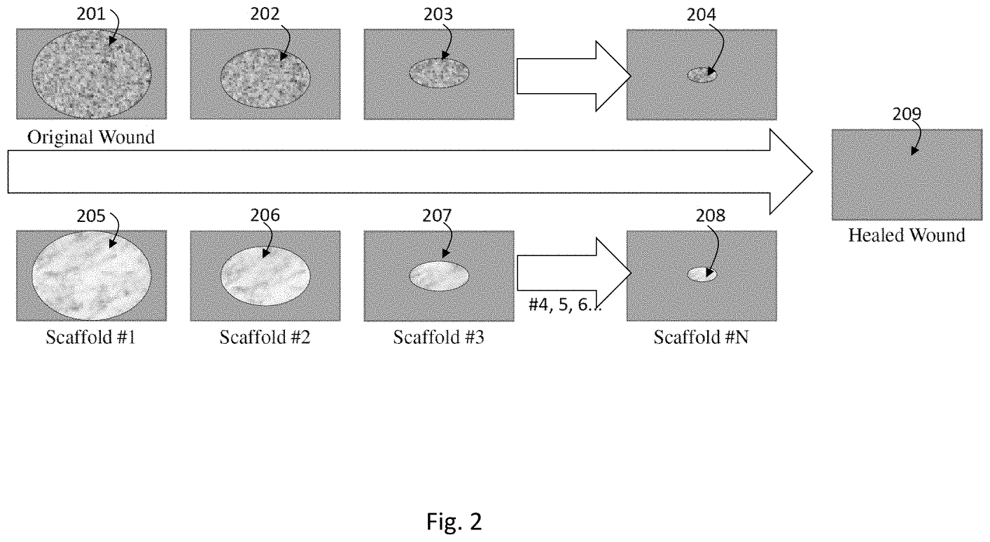

FIG. 2 illustrates the process of wound healing using sequential customized scaffolds. 201 shows the original wound and 205 shows the original scaffold. Wounds 202, 203 and 204 show the progressing healing of the wound and scaffolds 206, 207 and 208 show the corresponding scaffold for the different stages of the healed wound. Healed wound 209 illustrates completely healed wound. The arrows show wound healing progression from the original wound to the healed wound.

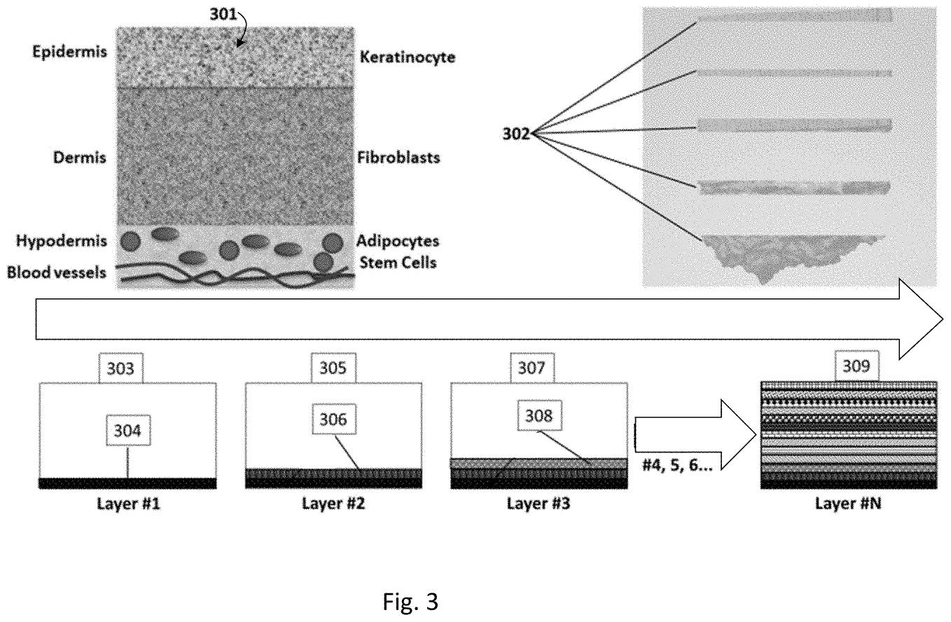

FIG. 3 illustrates the process to create multiple layers of scaffolds suitable for implantation. As shown, there are multiple layers of scaffold formation. Each layer comprises either matrix, cells, or both matrix and cells. Image 301 shows a picture of different layers of skin and the corresponding cells. Layers 302 are seen in side view of the wound highlighting the multiple sectioned layers of the 3D model for printing. The 3D models can be sectioned and modified using any CAD software to decide the number of layers. Wound scaffold 303 shows the wound scaffolds with first layer 304. Wound scaffold 305 shows the wound scaffolds with two layers, the second layer 306 on the first layer 304. Wound scaffold 307 shows wound scaffolds with three layers, with the third layer 308 on the second layer 306 that is on the first layer 304. Wound scaffold 309 shows wound scaffolds with multiple layers. Each of the layers can correspond to cells, matrices, therapeutics, growth factors, sensors, with each of them comprising of natural or synthetic components.

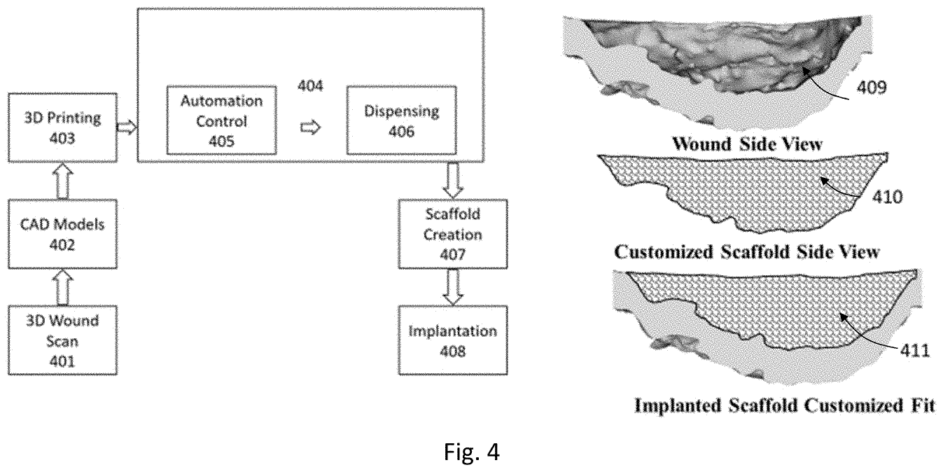

FIG. 4 illustrates one of the concepts of the process used for developing the customized implantable scaffolds for wound healing, which shows the schematic of 3D imaging, 3D printing, and hydrogel based scaffolds. A 3D wound scan can be performed (block 401), where an image or model can show the 3D wound scan. The model of the wound can be generated (block 402), which can include generating the CAD models developed from the wound. A printer can perform 3D printing (block 403), where any process of 3D printing of the wound molds can be performed. System components can be used for performing the 3D printing (block 404), where an automation controller (block 405), such as a computing system, can be configured for controlling dispensing of the scaffold material using valves (block 406) into the molds to develop the implantable scaffolds (block 407) for implantation in a wound 408 (block 408), optionally with sensors. Image 409 illustrates the wound side view, image 410 illustrates the scaffold side view and image 411 shows the implanted scaffold highlighting the customized fit.

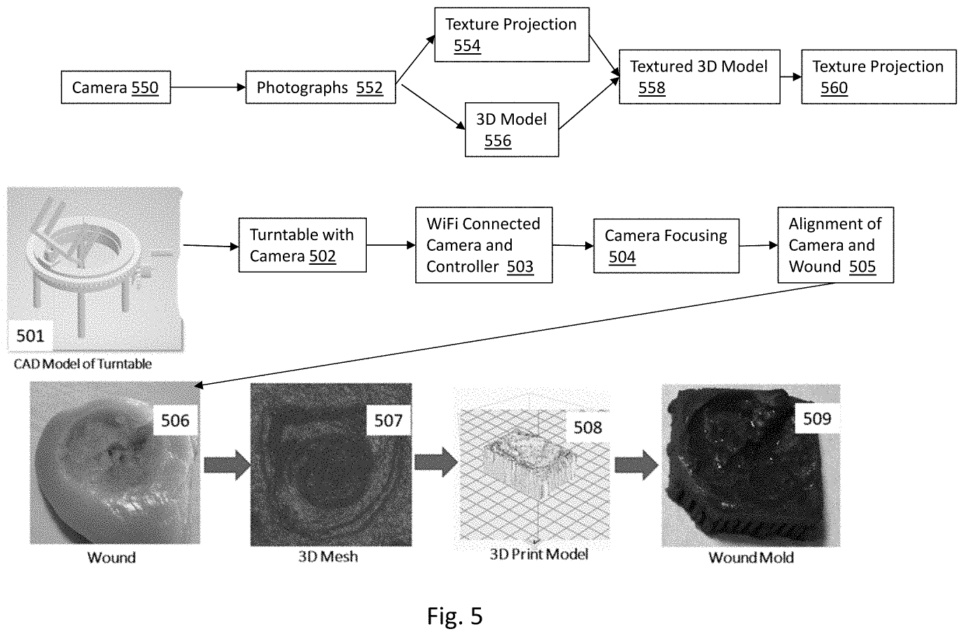

FIG. 5 provides an example of the process used to develop the 3D printed wound molds. As shown, the camera is provided (block 550), and photographs are prepared (block 552). Optionally, a texture projection can be made (block 554). A 3D model is prepared (block 556). A textured 3D model is prepared (block 558). Then, the 3D model is 3D printed (block 560).

Artificial skin glued to a Styrofoam base and grocery store bought chicken breasts were used to make different shapes and sizes of wounds. Next, a combination of liquid and coagulated makeup blood (Mehron Makeup) and food dyes (McCormick) was injected into the artificial skin and chicken breast based cuts to create the replica of deep cavity wounds for use in the experiments. FIG. 5 also shows the concept of photogrammetry to acquire 3D scan of the wounds to develop the 3D print model. First, in order to take 360 degree images, a rotating turntable system powered by a hand crank was designed in CAD (AutoDesk Inventor). The parts were printed using a 3D printer (Monoprice Maker Select V2). The camera was attached with a mounting bracket and a crosshair laser light served as guideline for optimal photo angle. The wounds were mounted in the center of the rotating turntable. The crosshair light was used to align the camera angle and at least 30 images were captured while the camera rotated 360 degrees around the wound using the turntable. The images were then transferred from the camera to the computer for post-processing before moving to generation of the CAD model. A cloud-connected software (ReCap Photo;AutoDesk) was used to create textured meshes and point clouds with high-resolution color images using the principles of photogrammetry. The resulting mesh was then exported in STL format and Autodesk Meshmixer was used to extrude the mesh. Slicing software (Cura) was then used to prepare the file for 3D printing using PLA. Monoprice Maker Select V2 3D printer was used to print the wound molds. FIG. 5 also shows the overall process to acquire images. A rotating turntable is provided (block 501) shown in CAD format is fabricated and used to attach a camera (block 502). A Wi-Fi connected camera and phone based operation is provided (block 503) and is used to focus camera (block 504) using a light based alignment system (block 505). The wound 506 is imaged to create the 3D mesh (block 507) which is used to develop the 3D print Model (block 508) to finally print the wound mold (block 509). Any camera with capability for close up or macro imaging can be used to take images. The alignment can be done using any kind of light source including LED and laser. The wound images can be post processed and merged to create the 3D model using any software capable of merging images and any CAD software can be used to visualize and modify the 3D model of the wounds before 3D, 4D, 5D, or 6D printing.

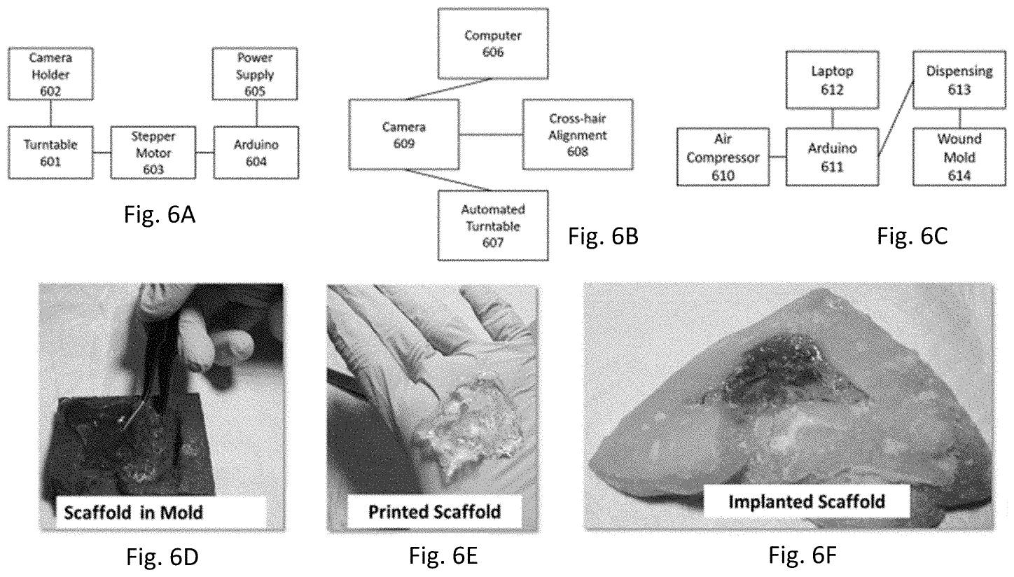

FIGS. 6A-6F provide an example of the fully automated process for imaging and development of the implantable scaffolds, which shows the photogrammetry based automated 3D imaging and development of implantable scaffolds. FIG. 6A shows the automated turn table, (block 601) with the camera holder containing camera (block 602). The motor is controlled using a stepper motor (block 603), an Arduino controller (block 604) and the power supply (block 605). The stepper motor controls the speed of rotation which is programmed by the Arduino board and software. The power supply helps in providing the desired output of power to the system. FIG. 6B shows the automated imaging system in action controlled by the computer (block 606) showing the computer with the automated turntable (block 608) showing the LED based cross-hair alignment (block 609) showing the mounted camera for acquiring the images. The monitor is used to ensure viewing of the images during acquisition and analysis. FIG. 6C shows the automated process for developing of the scaffold using the wound molds. The air compressor is provided (block 610) and is in communication with an Arduino controller (block 611) connected with solenoid valve to regulate the flow based on the pressure. The laptop is provided (block 610) for controlling the Arduino (block 611). The dispensing system is provided and operated (block 613) for the scaffold formation in the wound molds (block 614). FIG. 6D shows the scaffold after formation in the mold, FIG. 6E shows the developed scaffold and FIG. 6F shows the implanted scaffolds in the wound highlighting the perfect fit to the wound shape. Multiple dispensing systems can be arranged in parallel to develop scaffolds in a high throughput manner specific to each wound.

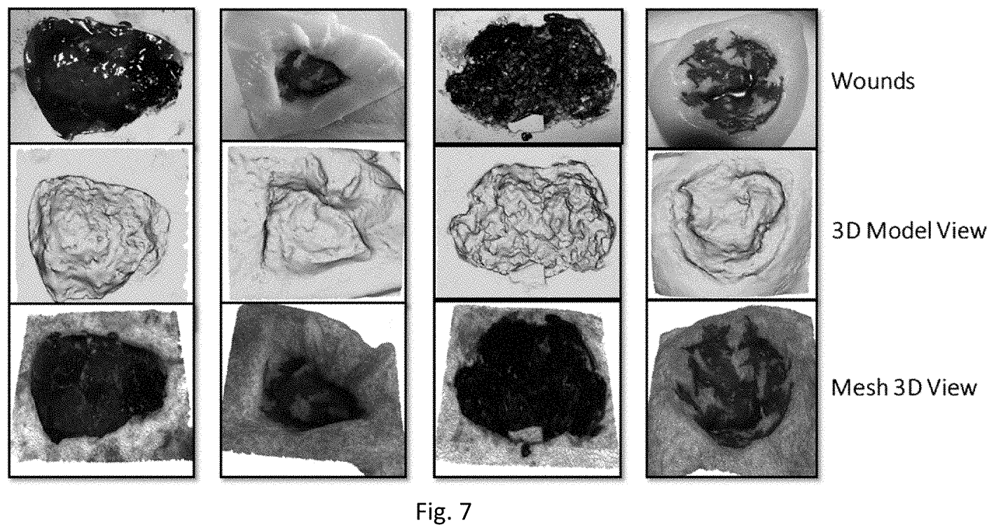

FIG. 7 shows images of the wounds, their corresponding 3D models, and mesh views indicating the ability to scan complex wound shapes and develop accurate 3D models. Examples of the automated photogrammetry based 3D imaging and model development are illustrated.

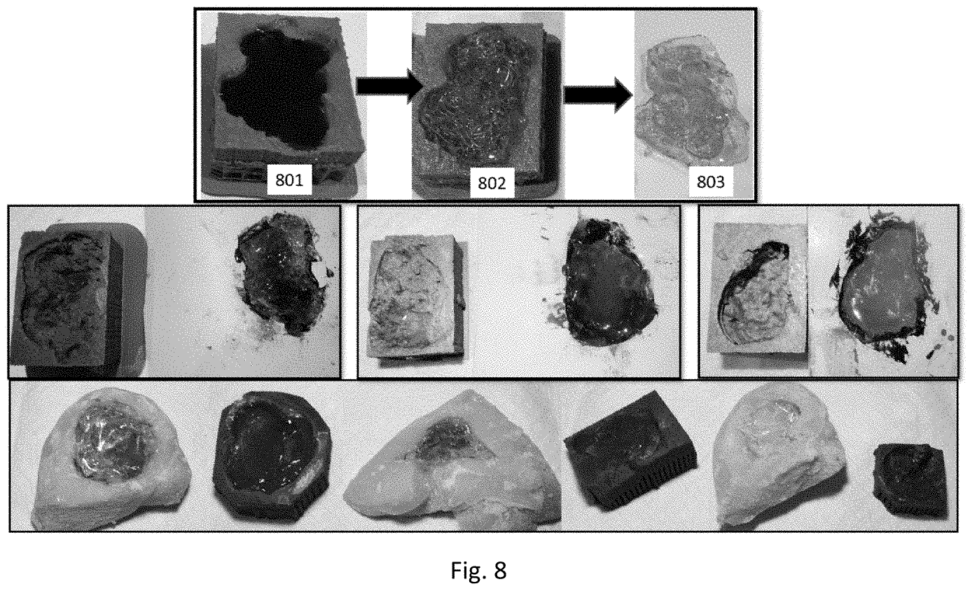

FIG. 8 shows further examples of the customized implantable scaffolds with perfect fit to the wounds developed using the wound molds (customized scaffolds for personalized wound fit). FIG. 8 shows the wound mold with liquid solution (block 801), converting to scaffolds (block 802) and the final formed scaffold (block 803). FIG. 8 also shows the various wounds and the customized and personalized scaffold fit irrespective of the morphology.

Scaffold formulations can include natural materials such as collagen, chitosan, and synthetic polymers such as polyethylene glycol diacrylate (PEGDA) and polyvinyl alcohol (PVA). Light curable crosslinking materials commonly used in the field of tissue engineering and bioprinting can also be readily used as wound scaffolds. Gelatin methacrylate (GelMA) is a natural gelatin-based bioprinting formulation that provides mammalian cells with the essential properties of their native environment while PEG-Fibrinogen (PF) is a semi-synthetic hydrogel made of the non-biological molecule polyethylene glycol (PEG) and natural molecule fibrinogen (PF) that provides high stability and bioactivity. GelMA crosslinks in presence of Lithium phenyl-2,4,6-trimethylbenzoylphosphinate (LAP) while PF crosslinks in presence of Eosin Y. The concentration of each of these hydrogels can decide the final stiffness of the implantable scaffold depending upon the wound type and location. Another commonly used scaffold which is already used in wound dressings is alginate and can be customized for implantable scaffolds. Alginate is a naturally occurring biopolymer obtained from brown seaweed. Alginates are biocompatible, non-toxic, with ability for high water uptake, biodegradable, and most importantly cost-effective. In order to form a gel, sodium alginate needs to come into contact with divalent ions such as calcium (Ca2+). As soon as sodium alginate is added to a solution of calcium chloride, a gel forms as the sodium ions are exchanged with calcium ions and the polymer becomes cross-linked. The longer the alginate is in contact with the calcium chloride solution, the more rigid the gel will become, as more cross-links with the calcium ions can be formed. Also, depending on the concentration of calcium ions, the gels are either thermo-reversible (low concentrations) or not (high concentrations). In order to make scaffolds, the air compressor output pressure was regulated at 20 psi and controlled using a 3-way solenoid valves connected to the syringe loaded with solution (alginate or calcium chloride). Drops of alginate solution were dispensed using a needle into the UV, ethanol, or autoclave sterilized wound molds to cross link and form the implantable scaffolds.

The scaffolds can be integrated with cells during the process to develop specific scaffolds based on the wound to be healed. The scaffolds can be also integrated with therapeutics, growth factors, oxygen generation molecules, active and passive sensors for monitoring of the local wound environment comprising of moisture, oxygen, pH, and therapeutic levels. The sensors can be electrical, optical, chemical, magnetic, or thermal. The sensors' data are observed by patients, nurses and doctors to decide on the next course of action for the wound treatment at the site or using an online system or a remotely enabled app. Finally, the wound can be covered with a conductive electrical or magnetic material of natural or synthetic origin to guide the treatment of wounds.

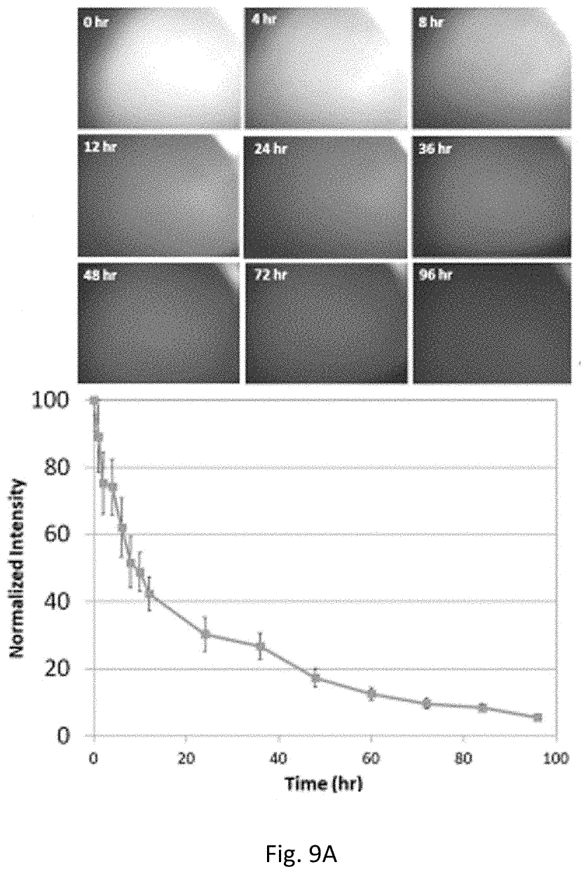

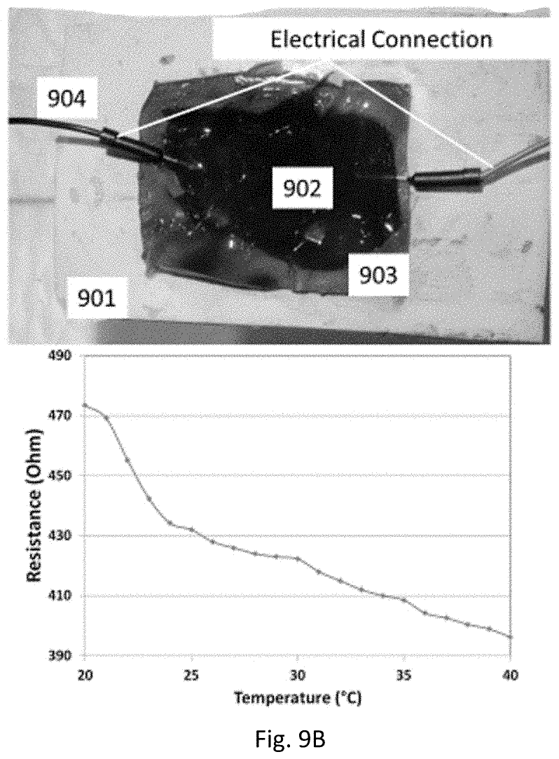

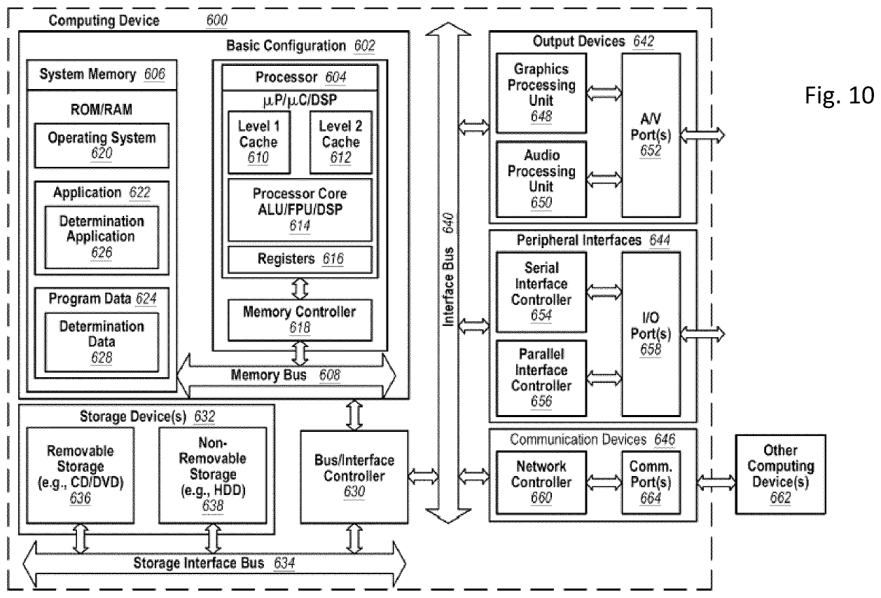

FIGS. 9A-9B shows example of monitoring of drug delivery and temperature. It is known that the local increase in temperature changes is indicative of infection in wounds. The native fluorescence of free chlorophyll molecules was chosen as the method to model drug delivery. Several spinach leaves were cut and placed in a mortar. Ethanol was added to just cover the leaves. A blender was then used to crush the leaves and release the chlorophyll from the thylakoid of the chloroplast. A filter paper was used to extract chlorophyll. The solution was tested under UV light to verify the presence of chlorophyll. The extracted chlorophyll was mixed with alginate. Alginate and chlorophyll mixture drops were dispensed into the calcium chloride solution to form scaffolds encapsulated with chlorophyll using the PLA mold of the deep wound cavity. The scaffolds were monitored to quantify the fluorescence changes as measure of drug release Images were acquired and analyzed using ImageJ. FIG. 9A shows the optical monitoring of drug release over 96 hours and the quantitative plot of the drug changes over the same period highlighting the successful release of drugs.

The unique property of pectin (extracted from fruits, gels similar to alginate in presence of Ca2+) for sensing temperature following electrical resistance changes was used to demonstrate the non-invasive monitoring. In order to integrate with the wound, a pectin film was made as a protective cover for the scaffold to mimic the skin layer. Once the film was formed, electrical probes were inserted on each side of the scaffold and the resistance was measured using an Arduino based ohmmeter. The temperature was varied by heating the scaffold and the changes in resistance were monitored. FIG. 9B shows the pectin film on top of the scaffold. The skin wound (901) shows the implanted scaffold (902) with the pectin film (903) and the electrical probes (904). FIG. 9B also shows the real-time monitoring of the temperature using the electrical resistance changes demonstrating the sensitivity of the pectin film. This allows detection of localized temperature changes which will be beneficial to monitor wound healing. Any increase in temperature will be indicative of infection in the wound while no change will indicate normal wound healing progression.

The implantable scaffolds can also be used to simulate drug delivery before implantation in the wounds. They can be used to optimize therapeutics concentration; drug release profiles in addition to choosing the optimal scaffold material, cells, and other components specific to the wound site and type. A database can also be built with each specific scaffold material, their properties, formulation information, and their detailed characterization to serve as a guide for scaffold generation for different wounds. Table 1 and 2 provide a summary of the time and cost for the developed system as a rapid and cost-effective solution for personalized implantable scaffolds for wound healing.

TABLE 1
Summary of Experimental Time for Developing Scaffolds

| Process | Time | |
|---|---|---|
| Imaging of Wound | 5 | min |
| Image Analysis | 30 min-1 hr | |
| 3D Printing of Molds | 2-4 | hr |

| | |
|---|---|
| Scaffold Generation | 30 min-1hr |

TABLE 2
Summary of Costs for the Developed System

| Requirements | Items | Total Cost |
|---|---|---|
| Equipment | Camera, Photogrammetry Software, 3D | $500: One |
| | printer, Automation Hardware | Time |
| Consumables | Filament, Alginate, Pectin, Calcium | $75~30- |
| | Chloride | 50 scaffolds |

In some embodiments, a system for preparing a personalized implantable wound healing scaffold is provided. The system can include: a 3D scanner or 3D imaging device (e.g. camera); a 3D model generator operably coupled with the 3D scanner, wherein the 3D model generator includes one or more processors and one or more non-transitory computer readable media storing instructions that in response to being executed by the one or more processors, cause the 3D model generator to perform operations; and a 3D, 4D, 5D, or 6D printer operably coupled with the 3D model generator. The operations can include: obtaining 3D scan data from the 3D scanner; generating a 3D image from the 3D scan data; and generating a 3D model from the 3D image. In some aspects, the printing can be by 3D printing.

In some embodiments, a mold caster is included in place of the 3D printer. However, the system can include both the 3D printer (or other printer) and the mold caster. In some aspects, the prosthesis is a partial mold with a 3D printed portion. For example, a mold can make a bulk member, and the 3D printing can provide the fine-tuned surfaces that interact with the wound when implanted. In some aspects, a 3D, 4D, 5D, or 6D printer operably coupled with the 3D model generator. In some aspects, a 3D mold caster is included, which also can be coupled with the 3D model generator to cast the generated 3D models.

In some embodiments, the system can include a sensor for real-time monitoring of the wound and/or wound healing scaffold having the sensor. The sensor can be placed proximal to the wound and implant when used. Also, the sensor can be embedded in the implant, such as during manufacture, which allows the sensor to be implanted with the implant. In some aspects, a monitor is operably coupled with the sensor for acquiring sensor data, saving the sensor data, and analyzing the sensor data.

In some embodiments, the system can include a turntable having an imaging device (camera) or a 3D scanner. The wound may be placed within the imaging region of the 3D scanner or the camera and the 3D scanner or camera can be rotated around the wound with the turntable. In some aspects, a motor can be coupled with the turntable, the motor being controlled by a controller computing system. The motor can allow for automated acquisition of the 3D data.

In some embodiments, the system can include a 3D, 4D, 5D, or 6D printing system having a reservoir of material, a pressure pump, a dispenser, and a mold receiver. The printing system can be any type of printing system that can form the structure of the implant by printing. The suitable compositions and equipment for such printing can also be provided.

In some embodiments, the 3D model generator is configured to generate 3D models of the wound. For example, the 3D model is a model of the wound to receive the wound healing scaffold. This allows for the wound healing scaffold to be prepared to precisely fit with the surface of the wound.

In some embodiments, the 3D model generator of the system is configured to generate 3D models of the wound and/or a scaffold to fit into the wound. The 3D model is prepared as a model of the wound healing scaffold adapted to fit into the wound.

In some embodiments, the 3D model generator is configured to generate 3D models of the wound and a scaffold to fit into the wound. The 3D model is a model of the wound and the wound healing scaffold is generated and adapted to fit into the wound.

In some embodiments, the system includes a lighting system adapted to illuminate the wound during the 3D scanning.

In some embodiments, a method of preparing a personalized implantable wound healing scaffold is provide. The method can include: scanning, with a 3D scanner, a wound; creating 3D images of the wound; creating 3D models of the wound from the 3D images; and printing, with a 3D, 4D, 5D, or 6D printer, a mold of the wound and/or an implantable wound healing scaffold.

In some embodiments, a method of preparing a personalized implantable wound healing scaffold is provided. The method can include: scanning, with a 3D scanner, a wound; creating 3D images of the wound; creating 3D models of the wound from the 3D images; printing, with a 3D, 4D, 5D, or 6D printer, a mold of the wound; and casting an implantable wound healing scaffold in the mold of the wound.

In some embodiments, a method of treating large and deep cavity wounds can include: 3D scanning of the wound; generation of point clouds and mesh of the wound; importing the mesh of the wound to a CAD software; extraction of the desired parts of wounds from the imported mesh; generating a CAD model of the wound; creation of wound mold; using the wound mold to develop exact replica of the wound in the form of a wound healing scaffold; integration with desired chemicals and biomolecules into the wound healing scaffold for treatment; integration of the wound healing scaffold with optical and electrical sensors for wound environment monitoring; and implantation of the wound healing scaffold.

In some embodiments, the methods can include integrating one or more with the wound healing scaffold: wound healing bioactive biological molecules, wound healing bioactive biomimetic molecules, wound healing bioactive synthetic molecules, cells, or sensors.

In some embodiments, the methods can include: implantation of the scaffold; monitoring of the scaffold for changes; removal of scaffold after certain days based on observed changes; rescanning of the wound to develop a second wound mold corresponding to the new morphology of the healing wound; and repeating the entire process till the desired level of healing is observed.

In some embodiments, a method of treating large and deep cavity wounds include: 3D scanning of the wound; generation of point clouds and mesh of the wound; importing the mesh of the wound to a CAD software, extraction of the desired parts of wounds from the imported mesh, generating CAD model of the wound; creation of wound mold; using the mold to develop exact replica of the wound with scaffold; integration with desired chemicals and biomolecules for treatment; integration of the scaffold with optical and electrical sensors for wound environment monitoring; implantation of the scaffold.

In some aspects, the method is to develop the replica of the wounds to aid in the development of healing. In some aspects, the method can include: implantation of the scaffold, monitoring of the scaffold for changes; removal of scaffold after certain days based on observed changes; rescanning of the wound to develop a second wound mold corresponding to the new morphology of the healing wound; repeating the entire process till the desired level of healing is observed.

In some aspects, the method can include resolving the 3D scans or 3D images to mimic the cell layers of the wound, skin, and the vasculature.

In some embodiments, the method can include: integrating a therapeutic (e.g. antimicrobials and antifungal drugs) with the scaffold to treat/prevent infection; testing drug release before implantation of the scaffold in the wound.

In some embodiments, the method can include: integrating cells of different layers of the skin and the vasculature in the scaffolds. The cells can be natural or synthetically modified cells.

In some embodiments, the method can include: mixing of different materials to create the scaffolds and match the stiffness of the desired wound area.

In some embodiments, the scaffold material could comprise of native components such as collagen, gelatin, chitosan, alginate, silk, elastin, laminin, fibronectin, hyaluronic acid, etc.) or synthetic polymers such as polyethylene glycol (PEG), polyethylene glycol diacrylate (PEGDA), polycaprolactone (PCL), polyvinyl alcohol (PVA), poly(d,l-lactide-co-glycolide) (PLGA), etc. The scaffolds can be developed from native, synthetic, modified (e.g. collagen methacrylate (ColMA) or gelatin methacrylate (GelMA), or a combination of those in varying ratio.

In some embodiments, the method can include: detecting changes in the local wound environment using embedded sensors; and comparing the readings from the sensor in the scaffolds with the readings of previous scaffolds and/or days to monitor local wound environment.

In some embodiments, the method can include: detecting changes in the local wound environment using superficial sensors; and comparing the readings from the sensor in the scaffolds with the readings of previous scaffolds and/or days to monitor local wound environment.

In some embodiments, the method can include: integration of electrical, temperature, light, moisture, oxygen, temperature, or pH sensitive components to monitor the local wound environment.

In some embodiments, the method can include: detecting changes in the local wound environment, transmitting the data actively or passively to a device (phone, computer) capable of receiving the transmission.

In some embodiments, the method can include: detecting changes in the local wound environment; transmitting the data actively or passively to a device (phone, computer) capable of receiving the transmission; using the data to decide the next course of wound treatment.

In some embodiments, the method can include: detecting changes in the local wound environment; transmitting the data actively or passively to an app capable of receiving the transmission; using the data to decide the next course of wound treatment.

In some embodiments, the 3D scanning method can include; generation of point clouds and mesh of the wound; importing the mesh of the wound to a CAD software, extraction of the desired parts of wounds from the imported mesh, generating CAD model for printing of the wound mold; 3D, 4D, 5D, or 6D printing of the wound mold; using the mold to develop exact replica of the wound with scaffold for treatment; implantation of the scaffold; integration of the scaffold with optical and electrical sensors for wound environment monitoring.

In some embodiment, the 3D scanning methods can comprise: Laser triangulation, Structured light 3D scanning technology, Photogrammetry, Contact-based 3D scanning technology, Laser pulse-based 3D scanning technology, Computerized Tomography, Single-photon emission computed tomography, Magnetic resonance imaging, Ultrasound imaging and terahertz spectroscopy.

In some embodiment, the wound molds for casting or printing and/or scaffolds to be prepared by casting or printing can be developed using 3D, 4D, 5D, or 6D printing methods based on inkjet, extrusion, and laser-based technologies. Examples include Stereolithography (SLA), Fused Deposition Modeling (FDM), Selective Laser Sintering (SLS), Digital Light Process (DLP), Multi Jet Fusion (MJF), PolyJet, Direct Metal Laser Sintering (DMLS), Electron Beam Melting (EBM), Solvent-based extrusion free forming (SEF), solvent-based extrusion (SBE), Laser-induced forward transfer (LIFT) bioprinting or a combination of the above.

In some aspects, the method is to develop the replica of the wounds to aid in the development of healing. In some aspects, the method can include: implantation of the scaffold, monitoring of the scaffold for changes; deciding the next mode of treatment for the wound; rescanning of the wound to develop a second scaffold with the desired treatment; repeating the entire process till the desired level of healing is observed.

In some embodiments, the wound molds can be made from natural or synthetic materials; can be biodegradable or sacrificial.

In some embodiments, the wound scaffold can comprise therapeutics such as natural or synthetic DNA, RNA, exosomes, proteins, antibacterial, antifungal, antiviral, bacteriophages, growth factors, cytokines, chemokines and other signaling molecules for cell and tissue growth. Examples include growth factors such as EGF, FGF, KGF, TGF-0, PDGF, VEGF, GM-CSF, CTGF, cytokines such as IL-1, IL-6, TNF and chemokines such as CXCL1, CXCL12, CCL2.

In some embodiments, the wound scaffold can include oxygen generation components such as calcium peroxide, magnesium peroxide, sodium percarbonate, hydrogen peroxide, fluorinated compounds such as perfluorodecalin and perflubron.

In some embodiments, the wound scaffold can include oxygen generation components from natural sources such as plants (chlorophyll, chloroplasts) and algae.

In some embodiments, the wound scaffold can include inorganic (metal, non-metal) or organic (polymeric, non-polymeric) particles.

In some embodiments, the wound scaffold can include magnetic or electrically conductive particles mixed or as a layer on top to provide pulse based therapy.

In some embodiments, a method for customizing a wound healing scaffold can include: locate wound; measure one or more dimensions of wound or provide a scale reference marker; 3D scan with wound; create 3D model; create 3D mold; and create the wound healing scaffold. In some aspects, the method can include: implanting wound healing scaffold into wound; and monitoring scaffold and wound.

In some embodiments, the methods may include removing a scaffold. Then, the method can include: 3D scan with wound; create 3D model; create 3D mold; and create the wound healing scaffold. In some aspects, the method can include: monitor scaffold and wound; remove scaffold; and discard scaffold.

In some embodiments, the methods can include developing the wound healing scaffold to have an epidermis layer with keratinocytes, a dermis layer with fibroblasts, and a base layer with adipocytes and/or stem cells.

In some embodiments, the methods can include determining one or more layers for the wound healing scaffold; preparing the one or more layers; and assembling the one or more layers to form the wound healing scaffold.

In some embodiments, a personalized implantable wound healing scaffold is provided, which can be prepared as described herein. The scaffold can include a 3D printed body having a shape of a wound of a subject, wherein the 3D printed body is biocompatible. The implant is customized to the wound so that the scaffold has a surface that matches a corresponding wound surface. In some aspects, the wound healing scaffold can include at least one of: wound healing bioactive biological molecules, wound healing bioactive biomimetic molecules, wound healing bioactive synthetic molecules, cells, or sensors. In some aspects, the wound healing scaffolds can be made from natural or synthetic materials, and can be biodegradable or sacrificial (non-biodegradable). In some aspects, the wound healing scaffold can comprise therapeutics such as natural and synthetic antibiotics/antifungals, antiviral, bacteriophages, growth factors, cytokines, chemokines and other signaling molecules for cell and tissue growth. Examples include growth factors such as EGF, FGF, KGF, TGF-β, PDGF, VEGF, GM-CSF, CTGF, cytokines such as IL-1, IL-6, TNF and chemokines such as CXCL1, CXCL12, CCL2. In some aspects, the wound healing scaffold can include oxygen generation components such as calcium peroxide, magnesium peroxide, sodium percarbonate, hydrogen peroxide, fluorinated compounds such as perfluorodecalin and perflubron. In some aspects, the wound healing scaffold can include oxygen generation components from natural sources such as plants (chlorophyll, chloroplasts) and algae. In some aspects, the wound healing scaffold can include inorganic (metal, non-metal) or organic (polymeric, non-polymeric) particles. In some aspects, the wound healing scaffold can include magnetic or electrically conductive particles mixed or as a layer on top to provide pulse based therapy.

Digital 3D models are used in a variety of industries, but there is more than one method to create them from a real wound. Photogrammetry and 3D scanning are the two primary methods for creating these models. When comparing 3D scanning to photogrammetry, two varieties are most common, which include laser and white light 3D scanning Laser scanning uses a laser to measure a wound's geometry and create the model through the data obtained. The laser beam is swept across the surface of the wound, and the device uses angle encoders of the beam projector and the return “time-of-flight” to calculate the location of each point in 3D space. Once all of the points are captured and recorded, a dense point cloud results. To capture a complete object, the laser scanner or object is moved, and the scan repeated. Optionally, additional software can connect the points to create a polygonal mesh for 3D modeling and design purposes.

White light 3D scanning utilizes a projector (often LCD) and one or more cameras to map an area or object. Light patterns are projected onto the surface, and the camera then records the surface by measuring where and how the light deforms around it. To ensure every angle is captured, the scanner is moved around the object, or the object is moved in front of the scanner. The result is a point cloud similar to what laser 3D scanning produces, with the same option of producing a polygonal mesh.

Photogrammetry (e.g., 3D image acquisition) is another method used to create 3D models of a wound. Instead of using active light sources, this technology uses photographs to gather data. Unlike the expensive machines needed for 3D scanning, photogrammetry only requires a camera, a computer, and specialized software. To create a 3D model using photogrammetry, photos are taken from a variety of angles to capture every part of the subject with overlap from picture to picture. This overlap is used by the software to align the photos appropriately. Once all of the images are taken, they are imported into the photogrammetry software, which aligns the pictures, plot data points, and calculates the distance and location of each point in the 3D space. The result is a 3D point cloud that can create a polygonal mesh, just like 3D scanning.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

FIG. 6 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include: providing a dataset having object data for an object and condition data for a condition; processing the object data of the dataset to obtain latent object data and latent object-condition data with an object encoder; processing the condition data of the dataset to obtain latent condition data and latent condition-object data with a condition encoder; processing the latent object data and the latent object-condition data to obtain generated object data with an object decoder; processing the latent condition data and latent condition-object data to obtain generated condition data with a condition decoder; comparing the latent object-condition data to the latent-condition data to determine a difference; processing the latent object data and latent condition data and one of the latent object-condition data or latent condition-object data with a discriminator to obtain a discriminator value; selecting a selected object from the generated object data based on the generated object data, generated condition data, and the difference between the latent object-condition data and latent condition-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A system for preparing a personalized implantable wound healing scaffold, the system comprising:
a 3D data acquisition unit including at least one of a 3D scanner or 3D imaging device;
a 3D model generator operably coupled with the 3D data acquisition unit, wherein the 3D model generator includes one or more processors and one or more non-transitory computer readable media storing instructions that in response to being executed by the one or more processors, cause the 3D model generator to perform operations, the operations comprising:
obtaining 3D data of a wound from the 3D data acquisition unit; and
generating a 3D model from the 3D data; and
a physical printer operably coupled with the 3D model generator.

2. The system of claim 1, wherein the physical printer is selected from a 3D, 4D, 5D, or 6D printer.

3. The system of claim 1, further comprising a 3D mold caster operably coupled with the 3D model generator.

4. The system of claim 1, further comprising:
a sensor for real-time monitoring of the wound, optionally a personalized implantable wound healing scaffold having the sensor; and
a monitor operably coupled with the sensor for acquiring sensor data, saving the sensor data, and analyzing the sensor data.

5. The system of claim 1, wherein the 3D model generator is configured to generate 3D models of the wound, and the 3D model is a model of the wound to receive the wound healing scaffold.

6. The system of claim 1, wherein the 3D model generator is configured to generate 3D models of the wound and/or personalized implantable wound healing scaffold to fit into the wound, and wherein the 3D model is a model of the wound and/or wound healing scaffold adapted to fit into the wound.

7. A method of preparing an implantable wound healing scaffold, the method comprising:
acquiring 3D data of a wound with a 3D acquisition unit that includes at least one of a 3D scanner or 3D imaging device;
generating a 3D model of the wound; and
forming the implantable wound healing scaffold in accordance with the 3D model of the wound.

8. The method of claim 7, wherein the forming of the implantable wound healing scaffold in accordance with the 3D model of the wound includes physically printing, with a 3D, 4D, 5D, or 6D printer, a mold of the wound and/or an implantable wound healing scaffold in accordance with the wound.

9. The method of claim 7, wherein the forming of the implantable wound healing scaffold in accordance with the 3D model of the wound includes casting an implantable wound healing scaffold in the mold of the wound.

10. The method of claim 1, further comprising:
generating at least one wound data structure of a point cloud or mesh of the wound;
importing the at least one wound data structure of the wound to a CAD software;
extracting desired parts of wounds from the imported mesh; and
generating a CAD model of the wound.

11. The method of claim 10, further comprising at least one of:
creating of wound mold, which includes a mold of the wound;
using the wound mold to develop exact replica of the wound in the form of a wound healing scaffold;
integrating at least one agent into the wound healing scaffold; or
integrating the wound healing scaffold with sensors for wound environment monitoring.

12. The method of claim 7, further comprising implanting the implantable wound healing scaffold in accordance into the wound.

13. The method of claim 12, further comprising:
monitoring the wound healing scaffold for changes;
removal of wound healing scaffold based on observed changes;
acquiring new 3D data of the wound by rescanning or reimaging of the wound to develop a second wound healing scaffold corresponding to the new morphology of the healing wound;
forming the second wound healing scaffold; and
implanting the second sound healing scaffold into the wound.

14. The method of claim 7, further comprising:
resolving the 3D data to mimic the layers of the wound, skin, and the vasculature;
integrating cells of different layers of the wound healing scaffold; and
forming a wound healing implant with an epidermis layer with keratinocytes, a dermis layer with fibroblasts, a base layer with adipocytes and/or stem cells and a vasculature structure with endothelial cells in the wound healing scaffold, wherein the cells can be natural or synthetically modified cells.

15. The method of claim 12, further comprising:
integrating a therapeutic composition with the wound healing scaffold to treat/prevent infection; and
releasing an active agent from the therapeutic composition before, during or after implantation of the scaffold in the wound.

16. The method of claim 7, further comprising forming the wound healing scaffold with a material with components selected from natural polymers selected from:
collagen, gelatin, chitosan, alginate, silk, elastin, laminin, fibronectin, hyaluronic acid, or combinations thereof; or
synthetic polymers selected from polyethylene glycol (PEG), polyethylene glycol diacrylate (PEGDA), polycaprolactone (PCL), polyvinyl alcohol (PVA), poly(d,l-lactide-co-glycolide) (PLGA), or combinations thereof; or
native, synthetic, modified collagen methacrylate (ColMA) or gelatin methacrylate, or a combination of those in varying ratio.

17. The method of claim 12, further comprising:
detecting changes in a local wound environment adjacent the wound healing scaffold;
transmitting data regarding the changes in the local wound environment actively or passively to a device and/or server and/or app and/or cloud capable of receiving the transmission; and
using the data to decide the next course of wound treatment.

18. The method of claim 7, further comprising performing at least one of: Laser triangulation, Structured light 3D scanning technology, Photogrammetry, Contact-based 3D scanning technology, Laser pulse-based 3D scanning technology, Computerized Tomography, Single-photon emission computed tomography, Magnetic resonance imaging, Ultrasound imaging, or terahertz spectroscopy.

19. The method of claim 7, further comprising:
developing a wound mold for casting or printing scaffolds, and using 3D, 4D, 5D, or 6D printing methods based on inkjet, extrusion, and laser-based technologies, selected from Stereolithography (SLA), Fused Deposition Modeling (FDM), Selective Laser Sintering (SLS), Digital Light Process (DLP), Multi Jet Fusion (MJF), PolyJet, Direct Metal Laser Sintering (DMLS), Electron Beam Melting (EBM), Solvent-based extrusion free forming (SEF), solvent-based extrusion (SBE), Laser-induced forward transfer (LIFT) bioprinting, or a combination of the above.

20. The method of claim 7, further comprising preparing the wound healing scaffold to include at least one of:
natural or synthetic DNA, RNA, exosomes, proteins, antibacterial therapeutics, antifungal therapeutics, antiviral, bacteriophages, growth factors, cytokines, chemokines and signaling molecules for cell and tissue growth, selected from the group consisting of: EGF, FGF, KGF, TGF-β, PDGF, VEGF, GM-CSF, CTGF, IL-1, IL-6, TNF, CXCL1, CXCL12, CCL2, or combinations thereof;
oxygen generation components selected from the group consisting of calcium peroxide, magnesium peroxide, sodium percarbonate, hydrogen peroxide, perfluorodecalin, perflubron, and combinations thereof;
oxygen generation components from natural sources;
inorganic metal or non-metal particles; or
organic polymeric or non-polymeric particles.

21. The method of claim 7, wherein the wound healing scaffold includes magnetic or electrically conductive particles mixed or as a layer on top to provide pulse based therapy.

22. The method of claim 7, further comprising:
determining one or more layers for the wound healing scaffold;
preparing the one or more layers; and
assembling the one or more layers to form the wound healing scaffold.

23. A method for customizing a wound healing scaffold, comprising:
locating a wound;
measuring one or more dimensions of the wound or providing a scale reference marker at the wound for imaging;
acquiring 3D data of the wound;
creating a 3D model of the wound from the 3D data;
creating a 3D mold from the 3D model; and
creating the wound healing scaffold in the 3D mold.

24. The method of claim 23, further comprising implanting the implantable wound healing scaffold in accordance into the wound.
